(19) United States
(12) Patent Application Publication
(10) Pub. No.: US 2024/0207600 A1
(43) Pub. Date: Jun. 27, 2024

(54) Real Time Heart Rate Monitoring for Close Loop Control and/or Artificial Pulse Synchronization of Implantable Ventricular Assist Devices

(71) Applicant: TC1 LLC, St. Paul, MN (US)

(72) Inventors: Daniel I. Harjes, Carlisle, MA (US); Brian Kimball, Medford, MA (US); Pritika Toutam, Plymouth, MN (US); Fujian Qu, San Jose, CA (US)

(73) Assignee: TC1 LLC, St. Paul, MN (US)

(21) Appl. No.: 18/088,079

(22) Filed: Dec. 23, 2022

Related U.S. Application Data
(60) Provisional application No. 63/310,674, filed on Feb. 16, 2022.

(51) Int. Cl. A61M 60/515; A61M 60/178; A61M 60/216; A61M 60/422; A61M 60/538

(52) U.S. Cl. CPC A61M 60/515; A61M 60/178; A61M 60/216; A61M 60/422; A61M 60/538

(57) ABSTRACT

Circulatory support systems and related methods are disclosed in which a ventricular assist device is controlled based on cardiac activity monitored via cardiogram electrodes. A circulatory support system includes a ventricular assist device, cardiogram electrodes, and a controller. The controller processes a cardiogram signal generated via the cardiogram electrodes to determine one or more physiological parameters indicative of an activity level and/or cardiac cycle timing, determines at least one operating parameter for the ventricular assist device based on the one or more physiological parameters, and controls operation of the ventricular assist device in accordance with the at least one operating parameter.

### CROSS REFERENCE TO RELATED APPLICATION DATA

The present application claims the benefit of U.S. Provisional Appln No. 63/310,674 filed Feb. 16, 2022; the full disclosure which is incorporated herein by reference in its entirety for all purposes.

### BACKGROUND

Ventricular assist devices, known as VADs, are implantable blood pumps used for both short-term (i.e., days, months) and long-term applications (i.e., years or a lifetime) where a patient's heart is incapable of providing adequate circulation, commonly referred to as heart failure or congestive heart failure. According to the American Heart Association, more than five million Americans are living with heart failure, with about 670,000 new cases diagnosed every year. People with heart failure often have shortness of breath and fatigue. Years of living with blocked arteries or high blood pressure can leave a heart too weak to pump enough blood to the body. As symptoms worsen, advanced heart failure develops.

A patient suffering from heart failure, also called congestive heart failure, may use a VAD while awaiting a heart transplant or as a long term destination therapy. In another example, a patient may use a VAD while their own native heart recovers. Thus, a VAD can supplement a weak heart (i.e., partial support) or can effectively replace the natural heart's function. VADs can be implanted in the patient's body and powered by an electrical power source inside or outside the patient's body.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In many embodiments, a circulatory support system includes a ventricular assist device (VAD) for a patient, electrocardiogram electrodes that are used to monitor cardiac activity of the patient, and a controller that controls operation of the VAD based on the cardiac activity of the patient. In some embodiments, the mean rotational speed of the VAD is varied based on an activity level of the patient determined by the controller based on the cardiac activity of the patient as measured by the electrocardiogram electrodes. As a result, the amount of blood pumped by the VAD can be better matched to the patient's needs for circulatory support over a larger range of activity levels as compared to operation of the VAD at a fixed rotational speed. In some embodiments, the VAD is operated in a pulsatile mode that produces a blood pressure pulse (via an increase in the rotational speed of the VAD) that is synchronized with a cardiac cycle of the patient. Synchronization of the blood pressure pulse with a cardiac cycle may achieve improved aortic pulse pressure, improved aortic valve opening/cycling, improved ventricle unloading and wall cycling (which may be beneficial when used in some cardiac recovery approaches), and/or enhanced washing of the ventricle, the VAD, and/or the aortic root. In many embodiments, the controller processes the output signal from the electrocardiogram electrodes to calculate heart rate, rhythm consistency, specific cardiac cycle timing events (current and previous cycle time points), and ECG-derived respiration rate. In some embodiments, heart rate, rhythm consistency, and respiration rate is used to infer patient activity level or arrhythmias, which adds diagnostic value (additional log file event data) but is also used in some embodiments in automatic closed loop pump speed control. For example, in some embodiments, increases in heart rate and respiration rate is used to trigger increased VAD rotational speed according to a pre-determined clinical algorithm. In some embodiments, cardiac cycle period is used to determine optimized artificial pulse parameters (amplitude, dwell time, etc.) in real time.

Thus, in one aspect, a circulatory support system includes a ventricular assist device (VAD), electrocardiogram (ECG) electrodes, and a controller. The VAD is configured for pumping blood from a ventricle of a heart of a patient to an artery to supplement or replace pumping of blood by the ventricle to the artery. The ECG electrodes are configured to generate an electrocardiogram signal. The controller includes at least one processor and a tangible memory device storing non-transitory instructions executable by the at least one processor to cause the at least one processor to: (a) process the electrocardiogram signal to determine one or more physiological parameters of the patient, wherein the one or more physiological parameters are indicative of an activity level and/or cardiac cycle timing of the patient; (b) determine at least one operating parameter for the ventricular assist device based on the one or more physiological parameters; and (c) control operation of the ventricular assist device in accordance with the at least one operating parameter.

In many embodiments, the controller controls operation of the VAD to adjust output of the VAD based on the activity level of the patient. For example, in many embodiments the at least one operating parameter comprises a reference rotational speed of the ventricular assist device. The one or more physiological parameters can include a heart rate of the patient. The tangible memory device can store a reference rotational speed lookup table that stores an array of reference rotational speeds for the ventricular assist device corresponding to an array of reference heart rates. The controller can be configured so that array of reference rotational speeds and/or the array of reference heart rates can be input into the tangible memory device by a medical professional. The tangible memory device can store data that defines a rotational speed for the ventricular assist device as a function of the heart rate of the patient. The controller can be configured so that the data that defines the rotational speed of the ventricular assist device as a function of the heart rate of the patient can be input into the tangible memory device by a medical professional. The reference rotational speed of the ventricular assist device can be a constant speed rotation rate for the ventricular assist device. In many embodiments: the reference rotational speed of the ventricular assist device is set to be equal to a first reference rotational speed at a first reference heart rate, the reference rotational speed of the ventricular assist device is set to be equal to a second reference rotational speed at a second reference heart rate, the second reference rotational speed is greater than the first reference rotational speed, and the second reference heart rate is greater than the first reference heart rate.

In many embodiments, the non-transitory instructions are executable by the at least one processor to cause the at least one processor to operate the ventricular assist device in an artificial pulse mode in which a rotational speed of the ventricular assist device is varied according to a repeating rotational speed pattern that is based on the reference rotational speed. Each cycle of the repeating rotational speed pattern can be synchronized with a respective cardiac cycle of the heart. The non-transitory instructions can be executable by the at least one processor to further cause the at least one processor to: (a) process the electrocardiogram signal to identify a time of occurrence of a reference point in a cardiac cycle of the heart, (b) determine a delay time based on a heart rate of the patient, and (c) begin a next cycle of the repeating rotational speed profile at a point in time that is the delay time from the time of occurrence of the reference point in the cardiac cycle of the heart. The delay time can be based on an input by a medical professional. The non-transitory instructions can be executable by the at least one processor to further cause the at least one processor to determine a rotational speed variation amplitude for the repeating rotational speed profile. The controller can use the rotational speed variation amplitude to control operation of the ventricular assist device so that a maximum rotational speed of the repeating rotational speed profile is greater than a minimum rotational speed of the repeating rotational speed profile by the rotational speed variation amplitude. In some embodiments: (a) the rotational speed variation amplitude is set to be equal to a first rotational speed variation amplitude at a first reference heart rate, (b) the rotational speed variation amplitude is set to be equal to a second rotational speed variation amplitude at a second reference heart rate, (c) the second rotational speed variation amplitude is greater than the first rotational speed variation amplitude, and (d) the second reference heart rate is greater than the first reference heart rate. Each cycle of the repeating rotational speed profile can be configured to generate a pressure pulse that is synchronized with ventricular systole of the respective cardiac cycle of the heart. Each cycle of the repeating rotational speed profile can be configured to generate a pressure pulse that is synchronized with ventricular diastole of the respective cardiac cycle of the heart. Each cycle of the repeating of the repeating rotational speed profile can be configured to generate a pressure pulse that is synchronized with the cardiac cycle of the heart so as to be aligned between ventricular systole and ventricular diastole.

In many embodiments, the non-transitory instructions are executable by the at least one processor to cause the at least one processor to process the electrocardiogram signal to detect an arrhythmia of the heart. The non-transitory instructions can be executable by the at least one processor to cause the at least one processor to and output an arrhythmia alarm in response to detecting the arrhythmia of the heart.

In many embodiments, the non-transitory instructions are executable by the at least one processor to cause the at least one processor to process the electrocardiogram signal to determine whether a heart rate of the patient is stable or unstable. The non-transitory instructions can be executable by the at least one processor to cause the at least one processor to employ a default rotation rate of the ventricular assist device in response a determination that the heart rate of the patient is unstable.

In many embodiments, the non-transitory instructions are executable by the at least one processor to cause the at least one processor to process the electrocardiogram signal to determine whether a heart rate of the patient has increased from a previous period. The non-transitory instructions can be executable by the at least one processor to cause the at least one processor to process the electrocardiogram signal to determine whether a respiration rate has increased from the previous period. The non-transitory instructions can be executable by the at least one processor to cause the at least one processor to increase a rotational rate of the ventricular assist device in response to determining that each of the heart rate and the respiration rate has increased from the previous period.

In many embodiments, the ECG electrodes are integrated into other components of the circulatory support system. For example, in some embodiments, the controller is configured to be implanted and the controller includes the electrocardiogram electrodes. Each of the ECG electrodes can form an external surface of the controller. The circulatory support system can include an implantable cardiac monitor that comprises the electrocardiogram electrodes. The circulatory support system can include an implantable transcutaneous energy transmission receiver that comprises the electrocardiogram electrodes. The circulatory support system can include an implantable transcutaneous energy transmission receiver that includes one of the electrocardiogram electrodes and the controller can be configured to be implanted and include one of the electrocardiogram electrodes.

For a fuller understanding of the nature and advantages of the present invention, reference should be made to the ensuing detailed description and accompanying drawings.

### BRIEF DESCRIPTION OF THE DRAWINGS

FIG. 1 is an illustration of a mechanical circulatory support system that includes a ventricular assist device (VAD) implanted in a patient's body, in accordance with many embodiments.

FIG. 2 is an exploded view of implanted components of the circulatory support system of FIG. 1.

FIG. 3 is an illustration of the VAD of FIG. 1 attached to the patient's heart to augment blood pumping by the patient's left ventricle.

FIG. 4 is a cross-sectional view of the VAD of FIG. 3.

FIG. 5 is an illustration of an embodiment of an electronics unit for the VAD of FIG. 3.

FIG. 6 is an illustration of a mechanical circulatory support system that includes implantable transcutaneous energy transmission system receiver, an implantable controller, and a ventricular assist device (VAD), in accordance with many embodiments.

FIG. 7 schematically illustrates a mechanical circulatory support system that includes electrocardiogram electrodes for measuring activity level and/or cardiac cycle timing of a patient, in accordance with many embodiments.

FIG. 8 illustrates an approach for controlling operation of a ventricular assist device and/or monitoring patent parameters using and an electrocardiogram signal, in accordance with embodiments.

FIG. 9 illustrates an example data flow in an approach for controlling operation of a ventricular assist device and/or monitoring patent parameters using and an electrocardiogram signal, in accordance with embodiments.

FIG. 10 illustrates example look-up table data for determining a mean ventricular assist device rotational speed for a measured heart rate, in accordance with embodiments.

FIG. 11 illustrates example activity level based variations in a rotational speed profile for a ventricular assist device and an approach for synchronization of the rotational speed profile with a cardiac cycle of the patient, in accordance with embodiments.

FIG. 12 schematically illustrates a portable computing device that can be employed in conjunction with a mechanical circulatory support system, in accordance with embodiments.

FIG. 13 and FIG. 14 illustrate an implantable circulatory support system controller that includes electrocardiogram electrodes and an amplifier for amplifying an ECG signal generated by the electrocardiogram electrodes, in accordance with embodiments.

FIG. 15 illustrates an implantable cardiac monitor that includes electrocardiogram electrodes for use in an circulatory support system, in accordance with embodiments.

FIG. 16 and FIG. 17 illustrate an implantable transcutaneous energy transmission system receiver that includes electrocardiogram electrodes and an amplifier for amplifying an ECG signal generated by the electrocardiogram electrodes, in accordance with embodiments.

FIG. 18 illustrates an implantable controller that includes one electrocardiogram electrode and an implantable transcutaneous energy transmission system receiver that includes one electrocardiogram electrode, in accordance with embodiments.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

In many embodiments described herein, a circulatory support system includes a ventricular assist device (VAD) for a patient, electrocardiogram electrodes that are used to monitor cardiac activity of the patient, and a controller that controls operation of the VAD based on the cardiac activity of the patient. For example, the mean rotational speed of the VAD can be varied based on an activity level of the patient determined by the controller based on the cardiac activity of the patient as measured by the electrocardiogram electrodes. As another example, the VAD can be operated in a pulsatile mode that produces a blood pressure pulse (via an increase in the rotational speed of the VAD) that is synchronized with a cardiac cycle of the patient.

Many existing ventricular assist devices essentially operate in constant speed mode in which the VAD rotor rotation rate is held constant and set by a clinician before patient discharge. Both centrifugal and axial flow pumps exhibit unique inverse relationships between flow and pressure at every speed as illustrated in a head-flow (HQ) curve for the pump. For a left ventricular assist device (LVAD), the average flow through the LVAD does increase somewhat in response to a drop in aortic pressure, which typically occurs as a result of an increase of the activity level of the patient. The increase in average flow through the LVAD produced by the reduced aortic pressure, however, is small compared to the native heart's ability to adjust cardiac output between rest and exercise states (Native ˜4-25 L/min, VAD˜3-6 L/min).

In many embodiments described herein, a controller controls operation of a VAD to automatically adjust the mean rotational speed of the VAD in real time in response to a significant change in physiologic demand. For example, in many embodiments the controller reduces the mean rotational speed of the VAD during sleep or rest to prevent ventricular suction events, thereby enabling the means rotational speed of the VAD to be set higher to provide enhanced profusion/support without fear of encountering suction events during sleep or rest. In many embodiments, the controller increase the mean rotational speed of the VAD when the patient is in a more active state (exercise response).

Aortic insufficiency is another notable adverse event that influences the rotational speed selected by the clinician for a VAD operated at a constant rotational speed. Aortic insufficiency can be caused by structural failure of the aortic valve after chronic support. It is hypothesized that lack of physiologic cycling of the aortic valve during prolonged closure of the aortic valve causes biomechanical deterioration and subsequent prolapse of the aortic valve. In an attempt to avoid the occurrence of aortic insufficiency, a clinician may set the rotational speed of the VAD to achieving periodic aortic valve opening rather than optimal hemodynamic support. In some embodiments, a controller controls operation of a VAD to periodically reduces the rotational speed of the VAD (at least during ventricular systole) to induce opening and closing of the aortic valve.

In some embodiments described herein, a controller controls operation of a VAD to operate in an artificial pulse mode that is synchronized with the cardiac cycle of the patient. In some existing circulatory support systems, a VAD is operated in an asynchronous manner in which the rotational speed of the VAD is varied in a repeated pattern on a fixed cycle time basis (e.g., one cycle every two seconds) regardless of the native cardiac cycle timing. While operation of a VAD in an asynchronous artificial pulse mode may provide several clinical benefits, such as inhibiting thrombus formation via enhanced washing of the VAD, the left ventricle, the aortic root, inhibiting the development of aortic insufficiency, operation of the VAD in an artificial pulse mode that is synchronized with the cardiac cycle may achieve improved aortic pulse pressure, improved aortic valve opening/cycling, improved ventricle unloading and wall cycling (which may be beneficial when used in some cardiac recovery approaches), and/or enhanced washing of the ventricle, the VAD, and/or the aortic root.

### Mechanically Circulatory Support Systems

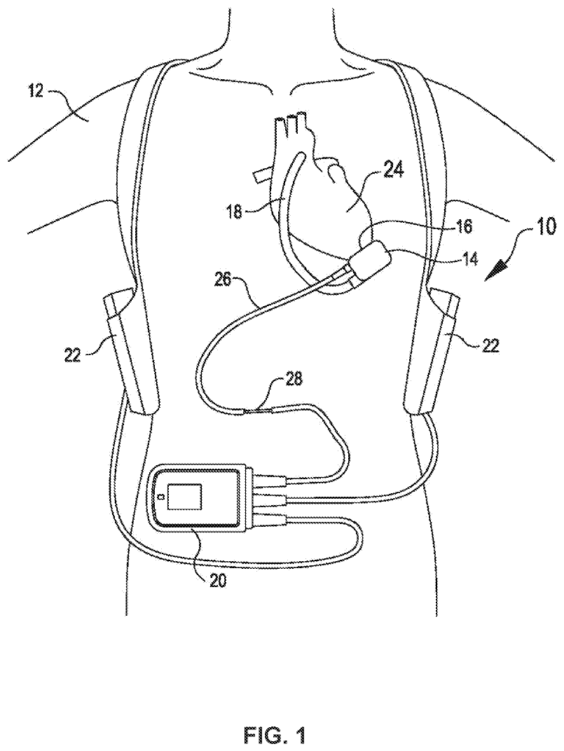

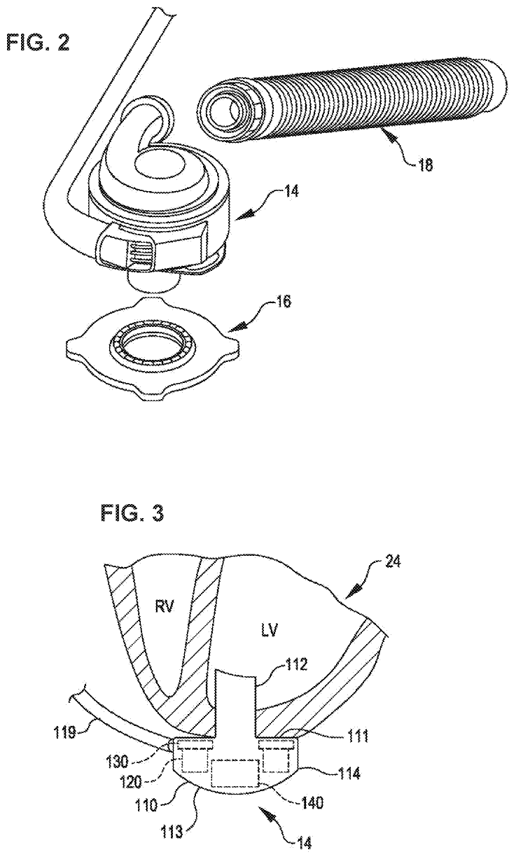

Referring now to the drawings, in which like reference numerals represent like parts throughout the several views, FIG. 1 is an illustration of a mechanical circulatory support system 10 that includes a ventricular assist device (VAD) 14 implanted in a patient's body 12. The mechanical circulatory support system 10 includes the VAD 14, a ventricular cuff 16, an outflow cannula 18, an external system controller 20, and power sources 22. A VAD 14 can be attached to an apex of the left ventricle, as illustrated, or the right ventricle, or a separate VAD can be attached to each of the ventricles of the heart 24. The VAD 14 can be capable of pumping the entire flow of blood delivered to the left ventricle from the pulmonary circulation (i.e., up to 10 liters per minute). Related blood pumps applicable to the present invention are described in greater detail below and in U.S. Pat. Nos. 5,695,471, 6,071,093, 6,116,862, 6,186,665, 6,234,772, 6,264,635, 6,688,861, 7,699,586, 7,976,271, 7,997,854, 8,007,254, 8,152,493, 8,419,609, 8,652,024, 8,668,473, 8,852,072, 8,864,643, 8,882,744, 9,068,572, 9,091,271, 9,265,870, and 9,382,908, all of which are incorporated herein by reference for all purposes in their entirety. With reference to FIG. 1 and FIG. 2, the VAD 14 can be attached to the heart 24 via the ventricular cuff 16, which can be sewn to the heart 24 and coupled to the VAD 14. In the illustrated embodiment, the output of the VAD 14 connects to the ascending aorta via the outflow cannula 18 so that the VAD 14 effectively diverts blood from the left ventricle and propels it to the aorta for circulation through the rest of the patient's vascular system.

FIG. 1 illustrates the mechanical circulatory support system 10 during battery 22 powered operation. A driveline 26 that exits through the patient's abdomen 28 connects the VAD 14 to the external system controller 20, which monitors system 10 operation. Related controller systems applicable to the present invention are described in greater detail below and in U.S. Pat. Nos. 5,888,242, 6,991,595, 8,323,174, 8,449,444, 8,506,471, 8,597,350, and 8,657,733, EP 1812094, and U.S. Patent Publication Nos. 2005/0071001 and 2013/0314047, all of which are incorporated herein by reference for all purposes in their entirety. The system 10 can be powered by either one, two, or more batteries 22. It will be appreciated that although the system controller 20 and power source 22 are illustrated outside/external to the patient body 12, the driveline 26, the system controller 20 and/or the power source 22 can be partially or fully implantable within the patient 12, as separate components or integrated with the VAD 14. Examples of such modifications are further described in U.S. Pat. Nos. 8,562,508 and 9,079,043, all of which are incorporated herein by reference for all purposes in their entirety.

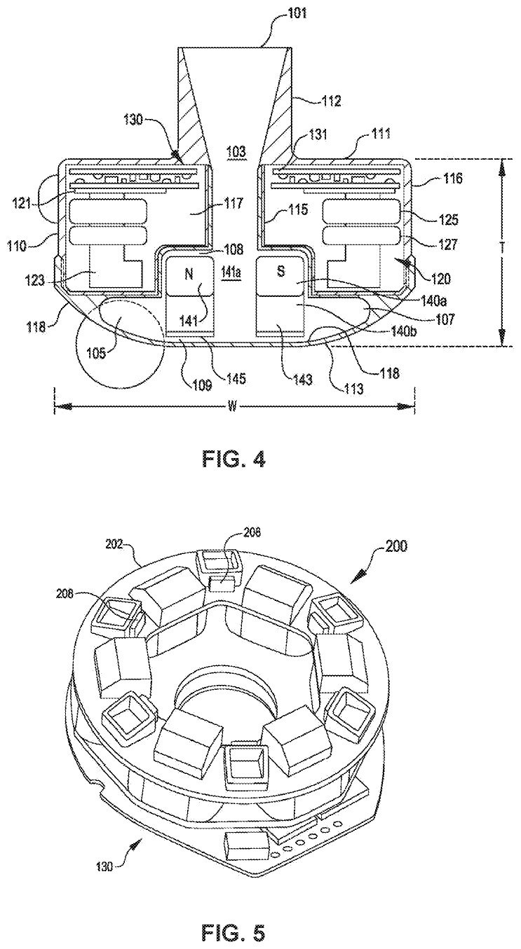

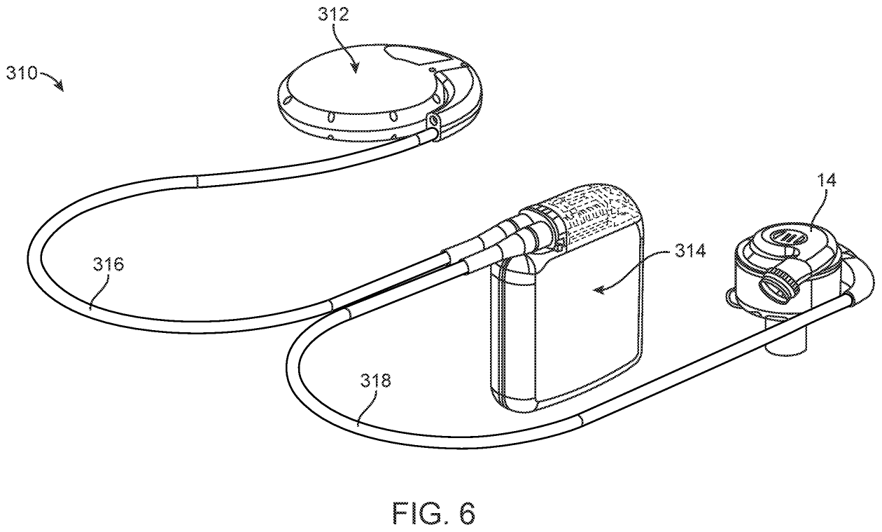

With reference to FIG. 3 and FIG. 4, the VAD 14 has a circular shaped housing 110 and is shown implanted within the patient 12 with a first face 111 of the housing 110 positioned against the patient's heart 24 and a second face 113 of the housing 110 facing away from the heart 24. The first face 111 of the housing 110 includes an inlet cannula 112 extending into the left ventricle LV of the heart 24. The second face 113 of the housing 110 has a chamfered edge 114 to avoid irritating other tissue that may come into contact with the VAD 14, such as the patient's diaphragm. To construct the illustrated shape of the puck-shaped housing 110 in a compact form, a stator 120 and electronics 130 of the VAD 14 are positioned on the inflow side of the housing toward first face 111, and a rotor 140 of the VAD 14 is positioned along the second face 113. This positioning of the stator 120, electronics 130, and rotor 140 permits the edge 114 to be chamfered along the contour of the rotor 140, as illustrated in at least FIG. 3 and FIG. 4, for example.

Referring to FIG. 4, the VAD 14 includes a dividing wall 115 within the housing 110 defining a blood flow conduit 103. The blood flow conduit 103 extends from an inlet opening 101 of the inlet cannula 112 through the stator 120 to an outlet opening 105 defined by the housing 110. The rotor 140 is positioned within the blood flow conduit 103. The stator 120 is disposed circumferentially about a first portion 140a of the rotor 140, for example about a permanent magnet 141. The stator 120 is also positioned relative to the rotor 140 such that, in use, blood flows within the blood flow conduit 103 through the stator 120 before reaching the rotor 140. The permanent magnet 141 has a permanent magnetic north pole N and a permanent magnetic south pole S for combined active and passive magnetic levitation of the rotor 140 and for rotation of the rotor 140. The rotor 140 also has a second portion 140b that includes impeller blades 143. The impeller blades 143 are located within a volute 107 of the blood flow conduit such that the impeller blades 143 are located proximate to the second face 113 of the housing 110.

The puck-shaped housing 110 further includes a peripheral wall 116 that extends between the first face 111 and a removable cap 118. As illustrated, the peripheral wall 116 is formed as a hollow circular cylinder having a width W between opposing portions of the peripheral wall 116. The housing 110 also has a thickness T between the first face 111 and the second face 113 that is less than the width W. The thickness T is from about 0.5 inches to about 1.5 inches, and the width W is from about 1 inch to about 4 inches. For example, the width W can be approximately 2 inches, and the thickness T can be approximately 1 inch.

The peripheral wall 116 encloses an internal compartment 117 that surrounds the dividing wall 115 and the blood flow conduit 103, with the stator 120 and the electronics 130 disposed in the internal compartment 117 about the dividing wall 115. The removable cap 118 includes the second face 113, the chamfered edge 114, and defines the outlet opening 105. The cap 118 can be threadedly engaged with the peripheral wall 116 to seal the cap 118 in engagement with the peripheral wall 116. The cap 118 includes an inner surface 118a of the cap 118 that defines the volute 107 that is in fluid communication with the outlet opening 105.

Within the internal compartment 117, the electronics 130 are positioned adjacent to the first face 111 and the stator 120 is positioned adjacent to the electronics 130 on an opposite side of the electronics 130 from the first face 111. The electronics 130 include circuit boards 131 and various components carried on the circuit boards 131 to control the operation of the VAD 14 (e.g., magnetic levitation and/or drive of the rotor) by controlling the electrical supply to the stator 120. The housing 110 is configured to receive the circuit boards 131 within the internal compartment 117 generally parallel to the first face 111 for efficient use of the space within the internal compartment 117. The circuit boards also extend radially-inward towards the dividing wall 115 and radially-outward towards the peripheral wall 116. For example, the internal compartment 117 is generally sized no larger than necessary to accommodate the circuit boards 131, and space for heat dissipation, material expansion, potting materials, and/or other elements used in installing the circuit boards 131. Thus, the external shape of the housing 110 proximate the first face 111 generally fits the shape of the circuits boards 131 closely to provide external dimensions that are not much greater than the dimensions of the circuit boards 131.

With continued reference to FIG. 4, the stator 120 includes a back iron 121 and pole pieces 123a-123f arranged at intervals around the dividing wall 115. The back iron 121 extends around the dividing wall 115 and is formed as a generally flat disc of a ferromagnetic material, such as steel, in order to conduct magnetic flux. The back iron 121 is arranged beside the control electronics 130 and provides a base for the pole pieces 123a-123f.

Each of the pole piece 123a-123f is L-shaped and has a drive coil 125 for generating an electromagnetic field to rotate the rotor 140. For example, the pole piece 123a has a first leg 124a that contacts the back iron 121 and extends from the back iron 121 towards the second face 113. The pole piece 123a can also have a second leg 124b that extends from the first leg 124a through an opening of a circuit board 131 towards the dividing wall 115 proximate the location of the permanent magnet 141 of the rotor 140. In an aspect, each of the second legs 124b of the pole pieces 123a-123f is sticking through an opening of the circuit board 131. In an aspect, each of the first legs 124a of the pole pieces 123a-123f is sticking through an opening of the circuit board 131. In an aspect, the openings of the circuit board are enclosing the first legs 124a of the pole pieces 123a-123f.

In a general aspect, the VAD 14 can include one or more Hall sensors that may provide an output voltage, which is directly proportional to a strength of a magnetic field that is located in between at least one of the pole pieces 123a-123f and the permanent magnet 141, and the output voltage may provide feedback to the control electronics 130 of the VAD 14 to determine if the rotor 140 and/or the permanent magnet 141 is not at its intended position for the operation of the VAD 14. For example, a position of the rotor 140 and/or the permanent magnet 141 can be adjusted, e.g., the rotor 140 or the permanent magnet 141 may be pushed or pulled towards a center of the blood flow conduit 103 or towards a center of the stator 120.

Each of the pole pieces 123a-123f also has a levitation coil 127 for generating an electromagnetic field to control the radial position of the rotor 140. Each of the drive coils 125 and the levitation coils 127 includes multiple windings of a conductor around the pole pieces 123a-123f. Particularly, each of the drive coils 125 is wound around two adjacent ones of the pole pieces 123, such as pole pieces 123d and 123e, and each levitation coil 127 is wound around a single pole piece. The drive coils 125 and the levitation coils 127 are wound around the first legs of the pole pieces 123, and magnetic flux generated by passing electrical current though the coils 125 and 127 during use is conducted through the first legs and the second legs of the pole pieces 123 and the back iron 121. The drive coils 125 and the levitation coils 127 of the stator 120 are arranged in opposing pairs and are controlled to drive the rotor and to radially levitate the rotor 140 by generating electromagnetic fields that interact with the permanent magnetic poles S and N of the permanent magnet 141. Because the stator 120 includes both the drive coils 125 and the levitation coils 127, only a single stator is needed to levitate the rotor 140 using only passive and active magnetic forces. The permanent magnet 141 in this configuration has only one magnetic moment and is formed from a monolithic permanent magnetic body 141. For example, the stator 120 can be controlled as discussed in U.S. Pat. No. 6,351,048, the entire contents of which are incorporated herein by reference for all purposes. The control electronics 130 and the stator 120 receive electrical power from a remote power supply via a cable 119 (FIG. 3). Further related patents, namely U.S. Pat. Nos. 5,708,346, 6,053,705, 6,100,618, 6,222,290, 6,249,067, 6,278,251, 6,351,048, 6,355,998, 6,634,224, 6,879,074, and 7,112,903, all of which are incorporated herein by reference for all purposes in their entirety.

The rotor 140 is arranged within the housing 110 such that its permanent magnet 141 is located upstream of impeller blades in a location closer to the inlet opening 101. The permanent magnet 141 is received within the blood flow conduit 103 proximate the second legs 124b of the pole pieces 123 to provide the passive axial centering force though interaction of the permanent magnet 141 and ferromagnetic material of the pole pieces 123. The permanent magnet 141 of the rotor 140 and the dividing wall 115 form a gap 108 between the permanent magnet 141 and the dividing wall 115 when the rotor 140 is centered within the dividing wall 115. The gap 108 may be from about 0.2 millimeters to about 2 millimeters. For example, the gap 108 can be approximately 1 millimeter. The north permanent magnetic pole N and the south permanent magnetic pole S of the permanent magnet 141 provide a permanent magnetic attractive force between the rotor 140 and the stator 120 that acts as a passive axial centering force that tends to maintain the rotor 140 generally centered within the stator 120 and tends to resist the rotor 140 from moving towards the first face 111 or towards the second face 113. When the gap 108 is smaller, the magnetic attractive force between the permanent magnet 141 and the stator 120 is greater, and the gap 108 is sized to allow the permanent magnet 141 to provide the passive magnetic axial centering force having a magnitude that is adequate to limit the rotor 140 from contacting the dividing wall 115 or the inner surface 118a of the cap 118. The rotor 140 also includes a shroud 145 that covers the ends of the impeller blades 143 facing the second face 113 that assists in directing blood flow into the volute 107. The shroud 145 and the inner surface 118a of the cap 118 form a gap 109 between the shroud 145 and the inner surface 118a when the rotor 140 is levitated by the stator 120. The gap 109 is from about 0.2 millimeters to about 2 millimeters. For example, the gap 109 is approximately 1 millimeter.

As blood flows through the blood flow conduit 103, blood flows through a central aperture 141a formed through the permanent magnet 141. Blood also flows through the gap 108 between the rotor 140 and the dividing wall 115 and through the gap 109 between the shroud 145 and the inner surface 108a of the cap 118. The gaps 108 and 109 are large enough to allow adequate blood flow to limit clot formation that may occur if the blood is allowed to become stagnant. The gaps 108 and 109 are also large enough to limit pressure forces on the blood cells such that the blood is not damaged when flowing through the VAD 14. As a result of the size of the gaps 108 and 109 limiting pressure forces on the blood cells, the gaps 108 and 109 are too large to provide a meaningful hydrodynamic suspension effect. That is to say, the blood does not act as a bearing within the gaps 108 and 109, and the rotor is only magnetically-levitated. In various embodiments, the gaps 108 and 109 are sized and dimensioned so the blood flowing through the gaps forms a film that provides a hydrodynamic suspension effect. In this manner, the rotor can be suspended by magnetic forces, hydrodynamic forces, or both.

Because the rotor 140 is radially suspended by active control of the levitation coils 127 as discussed above, and because the rotor 140 is axially suspended by passive interaction of the permanent magnet 141 and the stator 120, no magnetic-field generating rotor levitation components are needed proximate the second face 113. The incorporation of all the components for rotor levitation in the stator 120 (i.e., the levitation coils 127 and the pole pieces 123) allows the cap 118 to be contoured to the shape of the impeller blades 143 and the volute 107. Additionally, incorporation of all the rotor levitation components in the stator 120 eliminates the need for electrical connectors extending from the compartment 117 to the cap 118, which allows the cap to be easily installed and/or removed and eliminates potential sources of pump failure.

In use, the drive coils 125 of the stator 120 generates electromagnetic fields through the pole pieces 123 that selectively attract and repel the magnetic north pole N and the magnetic south pole S of the rotor 140 to cause the rotor 140 to rotate within stator 120. For example, the one or more Hall sensors may sense a current position of the rotor 140 and/or the permanent magnet 141, wherein the output voltage of the one or more Hall sensors may be used to selectively attract and repel the magnetic north pole N and the magnetic south pole S of the rotor 140 to cause the rotor 140 to rotate within stator 120. As the rotor 140 rotates, the impeller blades 143 force blood into the volute 107 such that blood is forced out of the outlet opening 105. Additionally, the rotor draws blood into VAD 14 through the inlet opening 101. As blood is drawn into the blood pump by rotation of the impeller blades 143 of the rotor 140, the blood flows through the inlet opening 101 and flows through the control electronics 130 and the stator 120 toward the rotor 140. Blood flows through the aperture 141a of the permanent magnet 141 and between the impeller blades 143, the shroud 145, and the permanent magnet 141, and into the volute 107. Blood also flows around the rotor 140, through the gap 108 and through the gap 109 between the shroud 145 and the inner surface 118a of the cap 118. The blood exits the volute 107 through the outlet opening 105, which may be coupled to an outflow cannula.

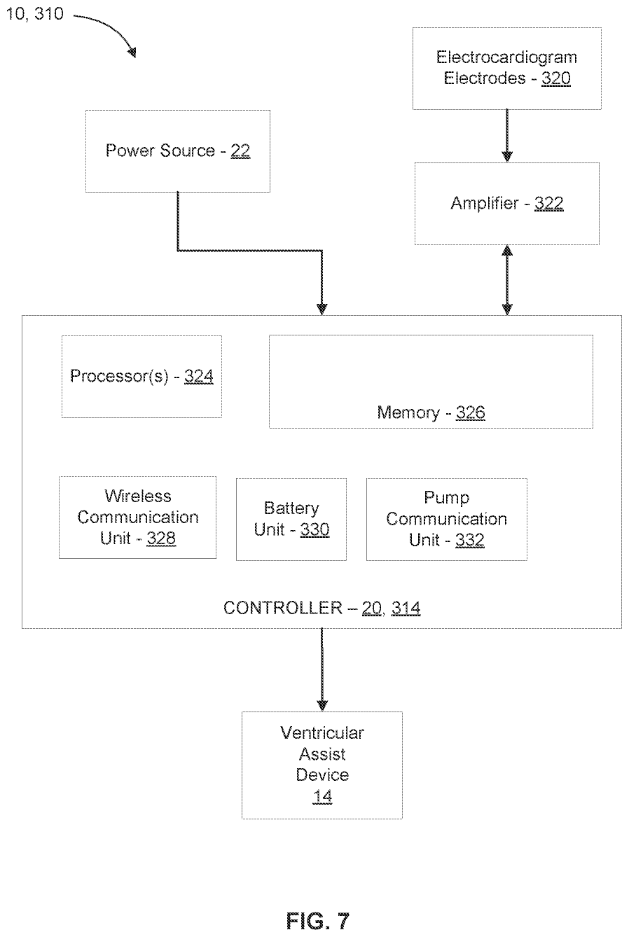

FIG. 5 shows a Hall Sensor assembly 200 for the VAD 14, in accordance with many embodiments. The Hall Sensor assembly 200 includes a printed circuit board (PCB) 202 and six individual Hall Effect sensors 208 supported by the printed circuit board 202. The Hall Effect sensors 208 are configured to transduce a position of the rotor 140 of the VAD 14. In the illustrated embodiment, the Hall Effect sensors 208 are supported so as to be standing orthogonally relative to the PCB 202 and a longest edge of each of the Hall Effect sensors 208 is aligned to possess an orthogonal component with respect to the surface of the PCB 202. Each of the Hall Effect sensors 208 generates an output voltage, which is directly proportional to a strength of a magnetic field that is located in between at least one of the pole pieces 123a-123f and the permanent magnet 141. The voltage output by each of the Hall Effect sensors 208 is received by the control electronics 130, which processes the sensor output voltages to determine the position and orientation of the rotor 140. The determined position and orientation of the rotor 140 is used to determine if the rotor 140 is not at its intended position for the operation of the VAD 14. For example, a position of the rotor 140 and/or the permanent magnet 141 may be adjusted, for example, the rotor 140 or the permanent magnet 141 may be pushed or pulled towards a center of the blood flow conduit 103 or towards a center of the stator 120. The determined position of the rotor 140 can also be used to determine rotor eccentricity or a target rotor eccentricity, which can be used as described in U.S. Pat. No. 9,901,666, all of which is incorporated herein by reference for all purposes in its entirety, to estimate flow rate of blood pumped by the VAD 14.

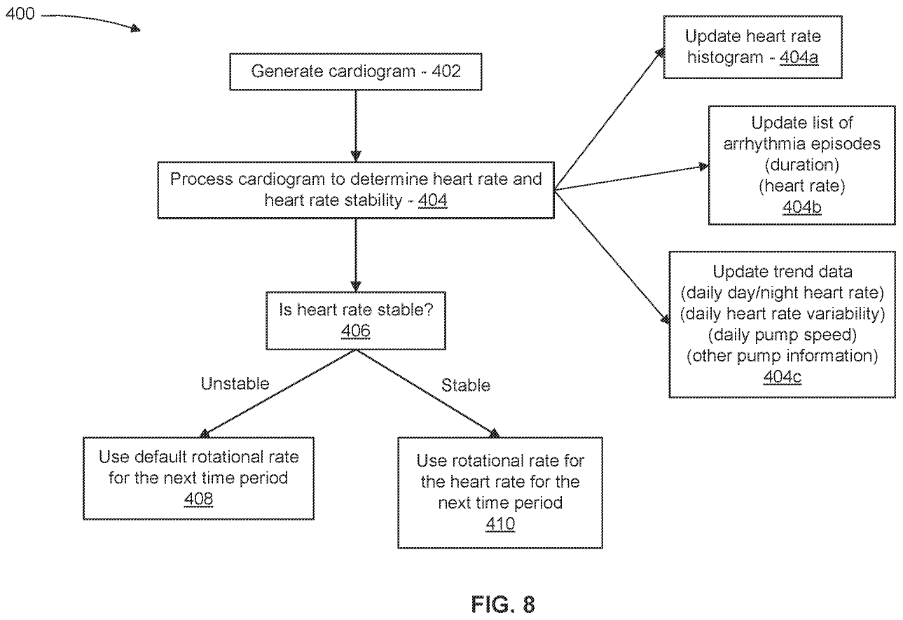

FIG. 6 shows a mechanical circulatory support system 310 that includes implantable transcutaneous energy transmission system (TETS) receiver 312, an implantable controller 314, the VAD 14, a first electrical cable 316, and a second electrical cable 318, in accordance with many embodiments. The implantable controller 314 is configured similar to the external controller 20, but is further configured to be implanted. The TETS receiver 312 can be, for example, a receiver, a resonator, and inductive coil or the like, for receiving transmitted electrical power used to power the circulatory support system 310. In some embodiments, the resonant frequency of the TETS receiver 312 is in a range of 100 kHz to 10 MHz.

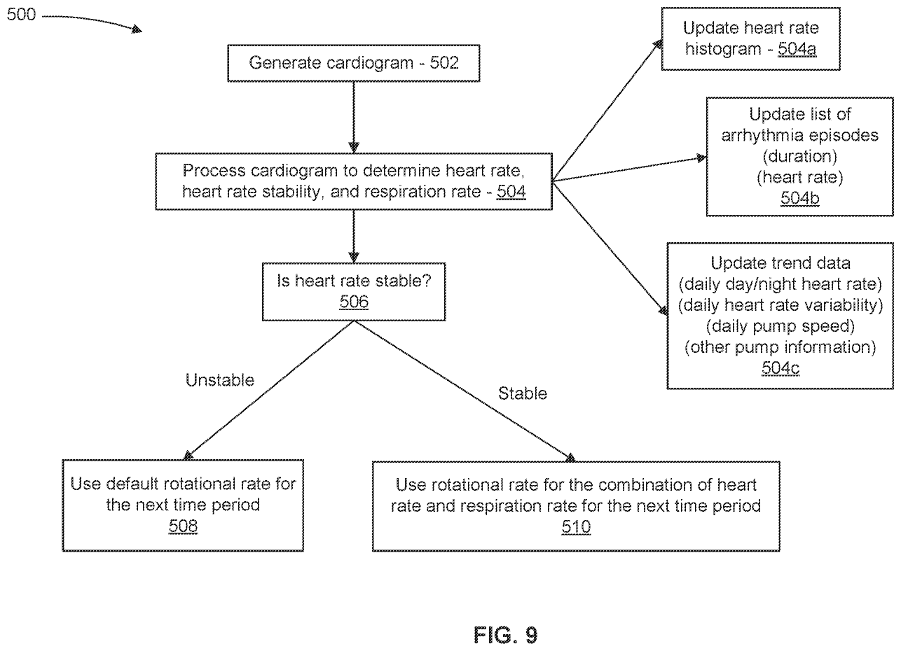

FIG. 7 schematically illustrates the mechanical circulatory support systems 10, 310. Each of the circulatory support systems 10, 310 include the power source 22, electrocardiogram electrodes 316, an amplifier 318, the controller 20, 314, and the VAD 14. In the circulatory support system 310, the power source 22 includes the TETS receiver 312. The controller 20, 314 include one or more processors 324, a memory storage device 325, a wireless communication unit 328, a battery unit 330, and a pump communication unit 3332. The memory storage device 326 can include any suitable type of memory and/or any suitable combination of types of memory. The memory storage device 326 can store suitable operating system instructions for the one or more processors 324. The memory storage device 326 can store suitable control data and instructions that are executable by the one or more processors 324 to process the output signal of the electrocardiogram electrodes 320, control the operation of the VAD 14 based on the output signal of the electrocardiogram electrodes 320 and the control data, and monitor the patent 12 including the functioning of the patient's heart 24 as described herein. Power received by the TETS receiver 312 is transmitted to the controller 20, 314 over the first electrical cable 316 and used to power the circulatory support system 10, 310. The battery unit 330 stores power received by the TETS receiver 312 for use in powering the circulatory support system 10, 310 when the TETS receiver 312 is not receiving power or is not receiving sufficient power for the operation of the circulatory support system 10, 310. The electrocardiogram electrodes 320 are configured to sense the electrical activity of the heart corresponding to the cardiac cycle. The output of the electrocardiogram electrodes 320 is amplified by the amplifier 322 and then processed by the one or more processors 324 as described herein to measure heart rate, respiration rate, and/or cardiac cycle timing. As described herein, the electrocardiogram electrodes 320 can be integrated into any suitable component(s) of the circulatory support system 10, 310, or can be part of a separate cardiac monitor device operatively coupled with the controller 20, 314. The wireless communication unit 328 can employ any suitable wireless communication protocol, such as any suitable WiFi communication protocol and/or any suitable Bluetooth communication protocol. The wireless communication unit 328 can be controlled by the one or more processors 324 to receive the control data for programming into the memory storage device 326 and to output notifications, alarms, heart rate histograms, and/or trend data to a suitable computing device, such as a suitable portable computing device (e.g., smartphone, tablet, and the like) as described herein. The pump communication unit 332 is controlled by the one or more processors 324 and transmits electrical signals to the VAD 14 over the second electrical cable 318 to power and control operation of the VAD 14.

### Cardiogram Based VAD Speed Control

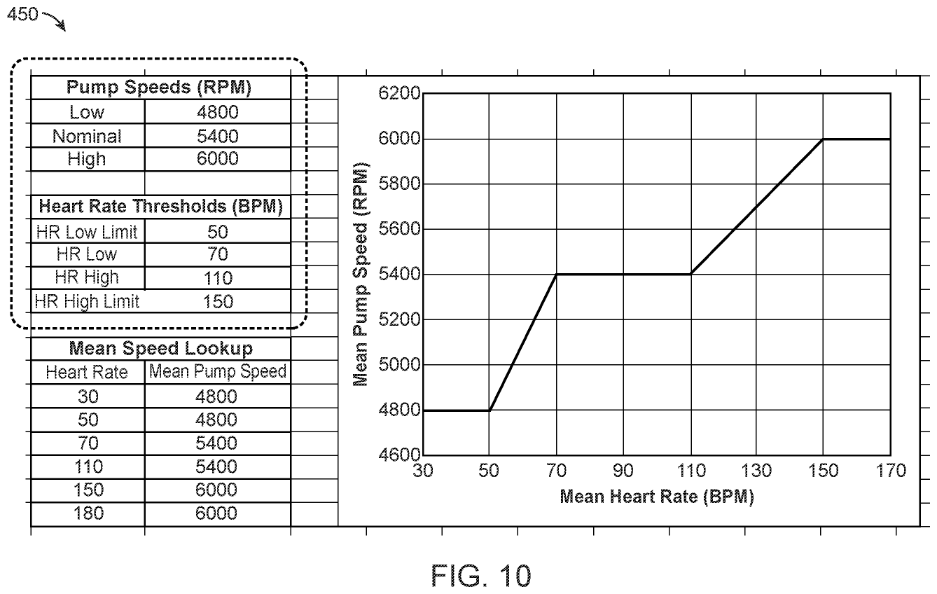

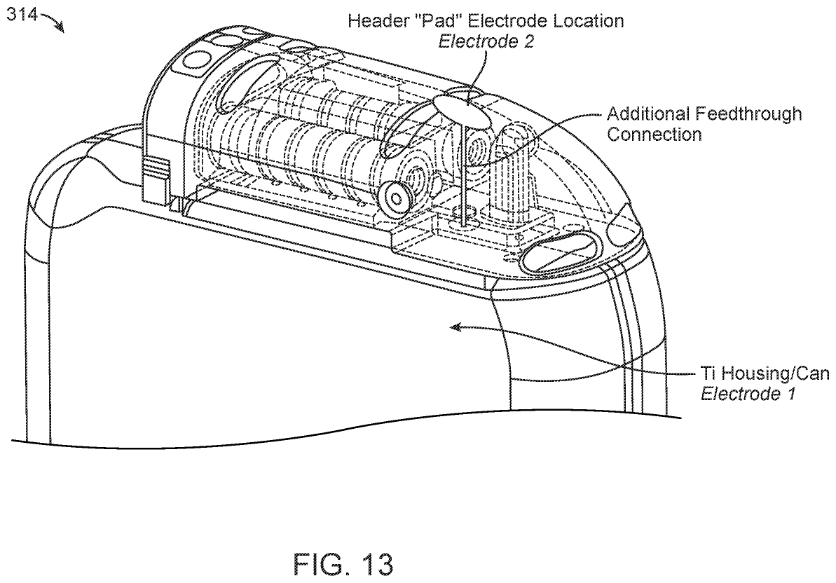

FIG. 8 illustrates an approach 400 for controlling operation of a ventricular assist device based on heart rate and monitoring patient parameters using a cardiogram generated via any suitable approach (e.g., via an electrocardiogram signal, via an impedance cardiogram signal). The approach 400 can be accomplished via any suitable circulatory assist system, such as the circulatory assist systems 10, 310 described herein. In act 402, a cardiogram for the patient is generated in real-time. The cardiogram can be generated using any suitable approach, such as via the electrocardiogram electrodes 320 and the amplifier 322. In act 404, the cardiogram is processed to determine heart rate and heart rate stability using any suitable known approach. Data generated in act 404 can be used to update a stored heart rate histogram (act 404a), update a list of arrhythmia episodes and store duration and the heart rate during the arrhythmia(s) (act 404b), and update stored trend data such as daily day/night heart rate, daily heart rate variability, daily pump speed, and/or other operational parameters of the VAD 14 (act 404c). In many embodiments, the cardiogram is processed by the controller 20, 314 using a suitable known approach. In act 404, a determination can be made whether the heart rate is within a low range of heart rates (e.g., less than 70 beats per minute), a medium range of heart rates (e.g., 70 to 110 beats per minute), or a high range of heart rates (e.g., greater than 110 beats per minute). Any suitable approach can be used to determine whether the heart rate is stable. For example, cardiac cycle-to-cycle timing can be evaluated to assess whether the heart rate is stable. If the heart rate is unstable, the VAD 14 is operated at a default rotational speed, such as a rotational speed for a medium range of heart rates (e.g., from 70 beats to 110 beats per minute in the example shown in FIG. 11) (act 408). If the heart rate is stable, the VAD 14 is operated during the next time period using a rotational rate that is a function of the heart rate (act 410). Any suitable approach can be used to determine the rotational rate, such as using a look-up table for the rotational rate (see example shown in FIG. 11) stored in the memory storage device 326 for the heart rate, or calculating the rotational rate for the heart rate using a suitable equation. The rotational rate for the next time period can be greater than, equal to, or less than the current rotational rate for the VAD 14 depending on whether the heart rate has increased, decreased, or has not changed from the heart rate used to control the VAD 14 for the current time period. In many embodiments, the process 400 is repeated periodically at a suitable time interval (e.g., every 10 to 30 seconds), which can be selected based on a suitable balance between a suitable number of cardiac cycles to analyze and a suitable response time. For example, when operating at a high rotational rate, a faster response time may be suitable to avoid a suction event. The rotational rate can also be recalculated at a relatively fast rate (e.g., every 5 to 10 seconds) and an algorithm used to look back in time over a longer interval (e.g., 30 to 60 seconds) to average the determined rotational rates over the longer interval for use during the next time period.

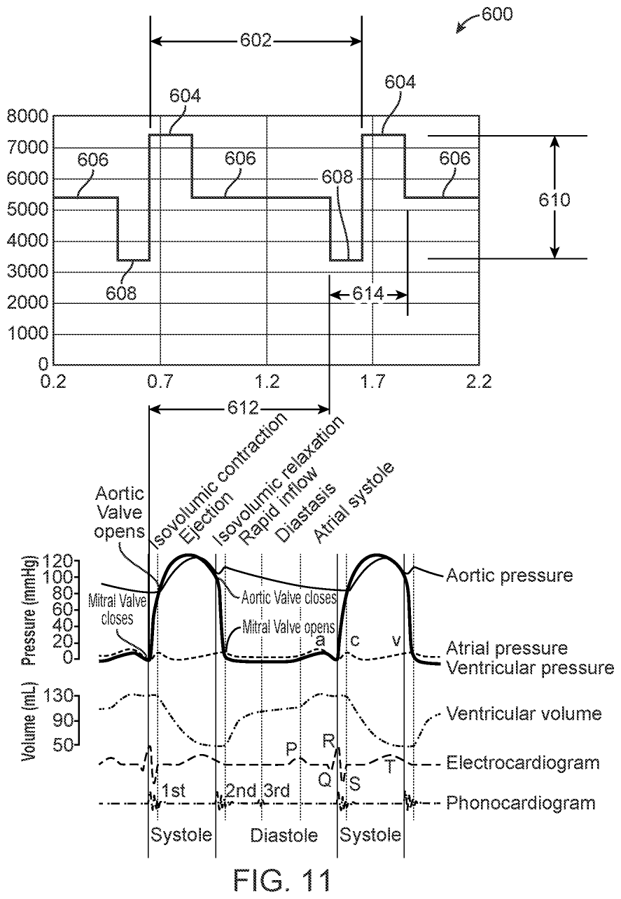

FIG. 9 illustrates an approach 500 for controlling operation of a ventricular assist device based on both heart rate and respiration rate and monitoring patient parameters using a cardiogram generated via any suitable approach (e.g., via an electrocardiogram signal, via an impedance cardiogram signal). The approach 500 can be accomplished via any suitable circulatory assist system, such as the circulatory assist systems 10, 310 described herein. In act 502, a cardiogram for the patient is generated in real-time. The cardiogram can be generated using any suitable approach, such as via the electrocardiogram electrodes 320 and the amplifier 322. In act 504, the cardiogram is processed to determine heart rate, heart rate stability, and respiration rate using any suitable known approach. Data generated in act 504 can be used to update a stored heart rate histogram (act 504a), update a list of arrhythmia episodes and store duration and the heart rate during the arrhythmia(s) (act 504b), and update stored trend data such as daily day/night heart rate, daily heart rate variability, daily pump speed, and/or other operational parameters of the VAD 14 (act 504c). In many embodiments, the cardiogram is processed by the controller 20, 314 using a suitable known approach. In act 504, a determination is made whether the heart rate is within a low range of heart rates (e.g., less than 70 beats per minute), a medium range of heart rates (e.g., 70 to 110 beats per minute), or a high range of heart rates (e.g., greater than 110 beats per minute). Any suitable approach can be used to determine whether the heart rate is stable. For example, cardiac cycle-to-cycle timing can be evaluated to assess whether the heart rate is stable. If the heart rate is unstable, the VAD 14 is operated at a default rotational speed, such as a rotational speed for a medium range of heart rates (e.g., from 70 beats to 110 beats per minute in the example shown in FIG. 11) (act 508). If the heart rate is stable, the VAD 14 is operated during the next time period using a rotational rate that is based on both the heart rate and the respiration rate (act 510). Any suitable approach can be used to determine the rotational rate, such as using a look-up table stored in the memory storage device 326 that defines the rotational rate (see example shown in FIG. 11) for the combination of the heart rate and the respiration rate, or calculating the rotational rate for the combination of the heart rate and the respiration rate using a suitable equation. The rotational rate for the next time period can be greater than, equal to, or less than the current rotational rate for the VAD 14 depending on if and how the heart rate and the respiration rate have changed from the heart rate and the respiration rate used to control the VAD 14 for the current time period. In many embodiments, the process 500 is repeated periodically at a suitable time interval (e.g., every 10 to 30 seconds), which can be selected based on a suitable balance between a suitable number of cardiac cycles to analyze and a suitable response time. For example, when operating at a high rotational rate, a faster response time may be suitable to avoid a suction event. The rotational rate can also be recalculated at a relatively fast rate (e.g., every 5 to 10 seconds) and an algorithm used to look back in time over a longer interval (e.g., 30 to 60 seconds) to average the determined rotational rates over the longer interval for use during the next time period.

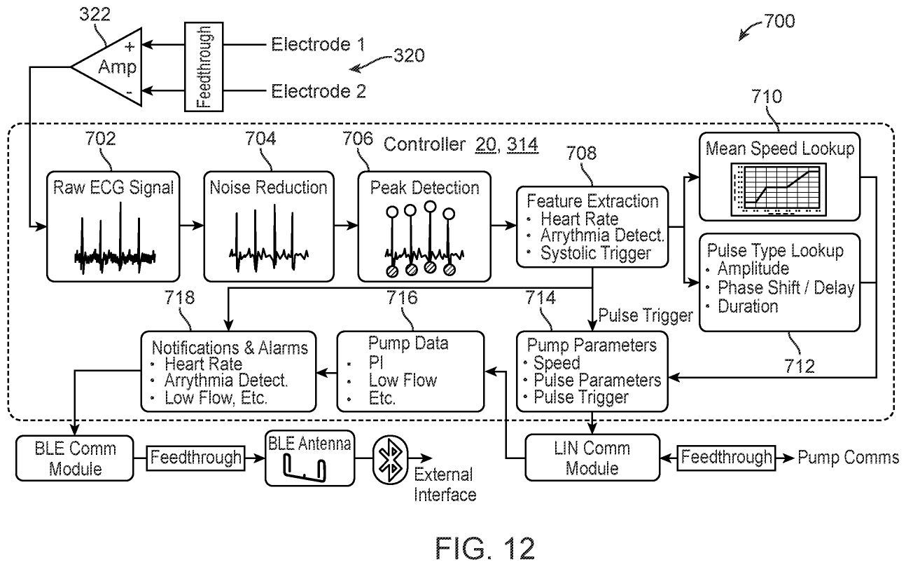

FIG. 10 illustrates example look-up data table 450 for determining a mean ventricular assist device rotational speed for a measured heart rate. The example look-up data table 450 can be used in the process 400. A similar look-up data table in which the rotation speed is a function of both heart rate and respiration rate can be used in the process 500.

FIG. 11 illustrates example activity level based variations in an example pulsatile rotational speed profile 600 for a ventricular assist device (e.g., VAD 14) and an approach for synchronization of the example pulsatile rotational speed profile 600 with the cardiac cycle of the patient. The example pulsatile rotational speed profile 600 repeats a rotational speed profile pattern 602. Each rotational speed profile pattern 602 includes a first segment 604, a second segment 606, and a third segment 608. The rotational speed of the VAD during the first segment 604 is greater than the rotational speed for the third segment 608 by a rotational speed pulse amplitude 610. The abrupt rotational speed increase from the third segment 608 of each respective rotational speed profile pattern 602 to the first segment 604 of the following rotational speed profile pattern 602 generates a pressure pulse in the blood flow output by the VAD. In the example rotational speed profile pattern 602, the rotational speed during the first segment 604 is greater than the rotational speed during the second segment 606 by 50 percent of the rotational speed pulse amplitude 610 and the rotational speed during the third segment 608 is less than the rotational speed during the second segment 606 by 50 percent of the rotational speed pulse amplitude 610.

In many embodiments, the controller 20, 314 controls operation of the VAD 14 in a pulsatile mode in which each respective rotational speed pattern (e.g., pattern 602) of pulsatile rotational speed profile (e.g., profile 600) is synchronized with a respective cardiac cycle of the heart 24. To accommodate the amount of time to process the ECG signal to detect the timing of respective occurrences of a suitable cardiac cycle trigger, a time delay 612 can be employed to determine how long after the occurrence of the cardiac cycle trigger to begin the next rotational speed pattern (e.g., pattern 602) so as to synchronized the start of the next rotational speed pattern with a desired phase of the next cardiac cycle. For example, in the illustrated embodiment, the occurrence of each R peak of the QRS complex of the ECG is employed as the cardiac cycle trigger and indicates the start of ventricular systole. The rotational speed pattern 602 can be configured to have a total time span equal to the time span of the cardiac cycle at the current heart rate. To start the next rotational speed pattern 602 at the start of ventricular systole, the delay 612 can be set to be equal to the time span of the cardiac cycle at the current heart rate. Variations in the delay 612 can be used to adjust the starting point of the next rotational speed pattern 602 to any desired phase of the next cardiac cycle. For example, the delay 612 and/or the mean rotational speed of the VAD 14 can be periodically set so as to reduce the output pressure of the VAD 14 during ventricular systole to induce cycling of the aortic valve.

In many embodiments, the controller 20, 314 adjusts the rotational speed pattern 602 based on the heart rate and/or the respiration rate of the patient. For example, the memory storage device 326 can store a lookup data table that defines the rotational speed pulse amplitude 610 as a function of the heart rate, a function of the respiration rate, or as a function of both the heart rate and the respiration rate. The controller can be configured so that array of reference rotational speeds and/or the array of reference heart rates can be input into the memory storage device 326 by a medical professional. As another example, the memory storage device 326 can store data that defines a rotational speed for the ventricular assist device as a function of the heart rate of the patient, such as data that defines an equation by which the rotational speed for the ventricular assist device is calculated as a function of the heart rate of the patient. The controller can be configured so that the data that defines the rotational speed of the ventricular assist device as a function of the heart rate of the patient can be input into the memory storage device 326 by a medical professional. Similarly, the relative time span of each of the first segment 604, the second segment 606, and/or the third segment 608 can be defined via a corresponding lookup data table stored in the memory storage device 326. For example, in the illustrated embodiment, the memory storage device 326 can store a lookup data table that defines a duration 614 of the third segment 608 and the following first segment 604 as a function of the heart rate and/or the respiration rate.

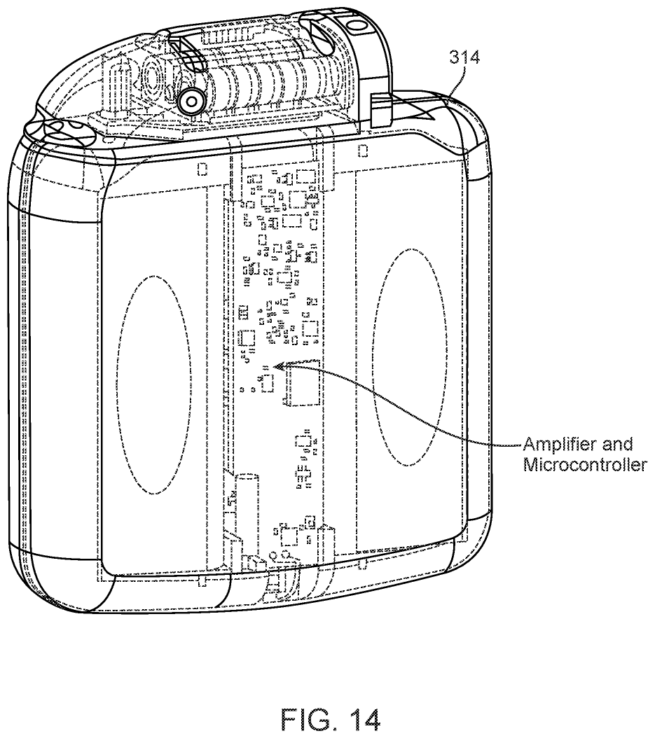

FIG. 12 illustrates an example data flow 700 that can be employed in either of the processes 400, 500. A voltage differential between the electrocardiogram electrodes 320 is amplified by the amplifier 322 and transferred to the controller 20, 314 as a raw ECG signal 702. The controller 20, 314 filters the raw ECG signal using a suitable band-pass filter (act 704) to reduce noise using a suitable known approach to produce a filtered ECG signal. The controller 20, 314 processes the filtered ECG signal to extract QRS peak timing data indicative of when Q, R, and S peaks of the QRS complex occur (act 706). In act 708, the controller 20, 314 processes the QRS peak timing data to extract features including heart rate, respiration rate, and systolic trigger timing data indicative of the start of systole in each cardiac cycle, and to detect the occurrence of arrhythmia(s). The controller 20, 314 accesses a mean speed lookup data table to determine a mean rotational speed for the VAD based on the heart rate and/or the respiration rate determined from the ECG signal (act 610). The controller 20, 314 can also access a pulse parameter lookup data table to determine pulse parameters (e.g., the amplitude 710, the delay 712, and the duration 714) based on the heart rate and/or the respiration rate as determined from the ECG signal (act 612). The controller 20, 314 controls the VAD 14 (act 614) based on the determined mean rotation speed for the VAD, the determined pulse parameters, and the cardiac cycle trigger (e.g., systolic trigger) to tailor the output of the VAD 14 to the activity level of the patient and to synchronize each pulsatile speed pattern 702 with a respective cardiac cycle of the heart 24. Stored operational data for the VAD 14 can be periodically updated (act 616). Notifications and alarms, such as high or low heart rate, arrhythmia, low pump flow, etc., can be generated and transmitted (act 618).

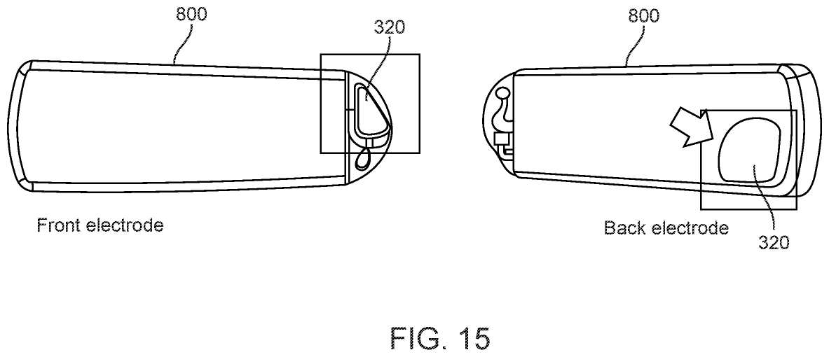

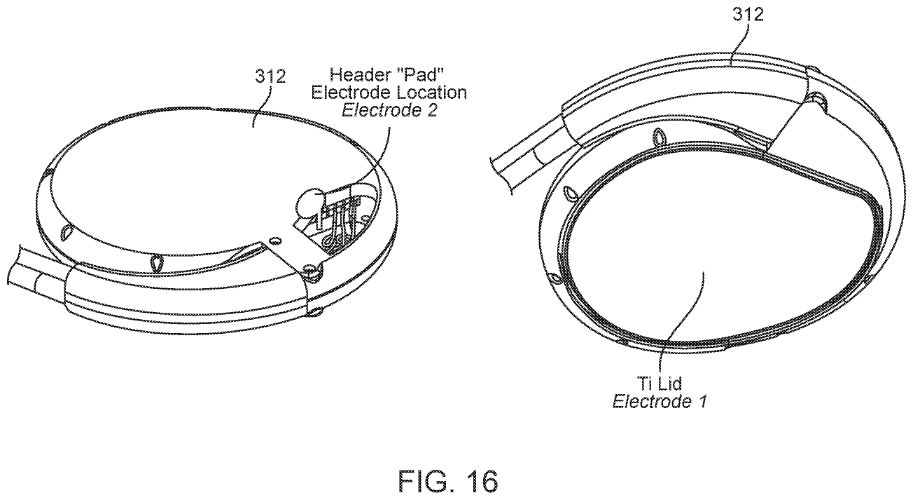

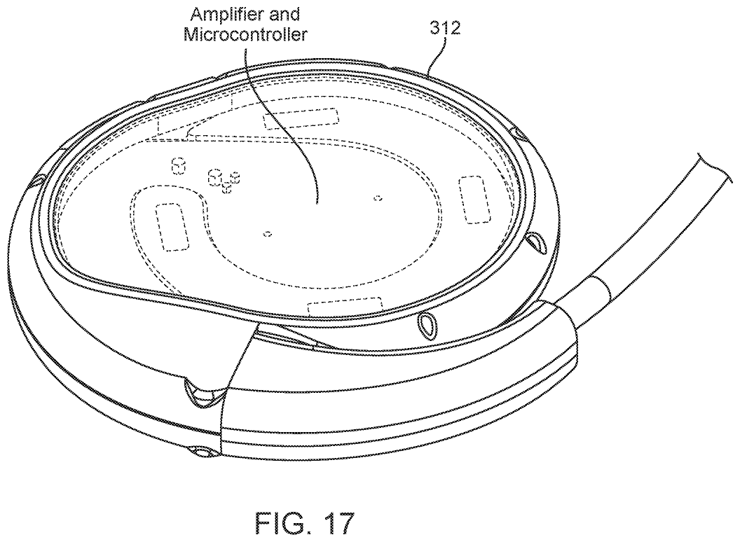

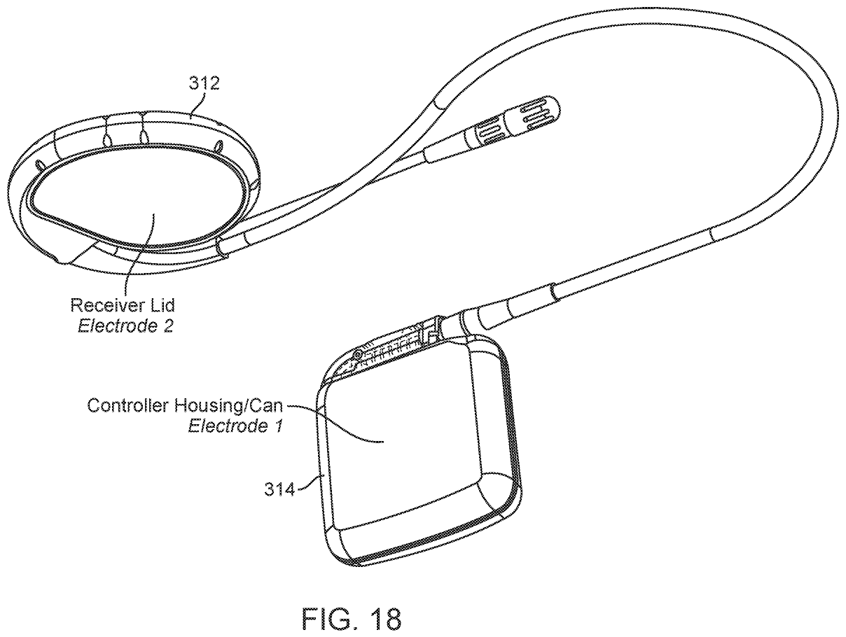

FIGS. 13 through 18 illustrate some example integrations of the ECG electrodes 320 into the circulatory support system 310. FIG. 13 illustrates the ECG electrodes 320 integrated into the controller 314 with a titanium housing of the controller 314 forming one of the electrodes 320 and another of the electrodes 320 being mounted to a non-conductive cover of the controller 314. FIG. 14 illustrates an example integration of the amplifier 322 into the controller 314. FIG. 15 illustrates an implantable cardiac monitor 800 that includes the ECG electrodes 320. FIG. 16 illustrates an example integration of the ECG electrodes 320 into the TETS receiver 312 wherein a titanium lid of the TETS receiver 312 forms one of the ECG electrodes 320 the other ECG electrode being mounted to a non-conductive housing portion of the opposite side of the TETS receiver 312. FIG. 17 illustrates an example integration of the amplifier 322 into the TETS receiver 312. FIG. 18 illustrates an example integration of the ECG electrodes into the circulatory support system 310 wherein a titanium lid of the TETS receiver 312 forms one of the ECG electrodes 320 and a titanium housing of the controller 314 forms the other of the ECG electrodes 320.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

What is claimed is:

1. A circulatory support system comprising:
a ventricular assist device configured for pumping blood from a ventricle of a heart of a patient to an artery to supplement or replace pumping of blood by the ventricle to the artery;
electrocardiogram electrodes configured to generate an electrocardiogram signal; and
a controller comprising at least one processor and a tangible memory device storing non-transitory instructions executable by the at least one processor to cause the at least one processor to:
process the electrocardiogram signal to determine one or more physiological parameters of the patient, wherein the one or more physiological parameters are indicative of an activity level and/or cardiac cycle timing of the patient;
determine at least one operating parameter for the ventricular assist device based on the one or more physiological parameters; and
control operation of the ventricular assist device in accordance with the at least one operating parameter.

2. The circulatory support system of claim 1, wherein the at least one operating parameter comprises a reference rotational speed of the ventricular assist device.

3. The circulatory support system of claim 2, wherein:
the one or more physiological parameters comprise a heart rate of the patient; and
the tangible memory device stores a reference rotational speed lookup table that stores an array of reference rotational speeds for the ventricular assist device corresponding to an array of reference heart rates.

4. The circulatory support system of claim 3, wherein the controller is configured so that array of reference rotational speeds and/or the array of reference heart rates can be input into the tangible memory device by a medical professional.

5. The circulatory support system of claim 2, wherein:
the one or more physiological parameters comprise a heart rate of the patient; and
the tangible memory device stores data that defines a rotational speed for the ventricular assist device as a function of the heart rate of the patient.

6. The circulatory support system of claim 5, wherein the controller is configured so that the data that defines the rotational speed of the ventricular assist device as a function of the heart rate of the patient can be input into the tangible memory device by a medical professional.

7. The circulatory support system of claim 2, wherein the reference rotational speed of the ventricular assist device is a constant speed rotation rate for the ventricular assist device.

8. The circulatory support system of claim 2, wherein:
the reference rotational speed of the ventricular assist device is set to be equal to a first reference rotational speed at a first reference heart rate;
the reference rotational speed of the ventricular assist device is set to be equal to a second reference rotational speed at a second reference heart rate;
the second reference rotational speed is greater than the first reference rotational speed; and
the second reference heart rate is greater than the first reference heart rate.

9. The circulatory support system of claim 2, wherein the non-transitory instructions are executable by the at least one processor to cause the at least one processor to operate the ventricular assist device in an artificial pulse mode in which a rotational speed of the ventricular assist device is varied according to a repeating rotational speed profile that is based on the reference rotational speed.

10. The circulatory support system of claim 9, wherein each cycle of the repeating rotational speed profile is synchronized with a respective cardiac cycle of the heart.

11. The circulatory support system of claim 10, wherein the non-transitory instructions are executable by the at least one processor to further cause the at least one processor to:
process the electrocardiogram signal to identify a time of occurrence of a reference point in a cardiac cycle of the heart;
determine a delay time based on a heart rate of the patient; and
begin a next cycle of the repeating rotational speed profile at a point in time that is the delay time from the time of occurrence of the reference point in the cardiac cycle of the heart.

12. The circulatory support system of claim 11, wherein the delay time can be set via an input by a medical professional.

13. The circulatory support system of claim 10, wherein the non-transitory instructions are executable by the at least one processor to further cause the at least one processor to determine a rotational speed variation amplitude for the repeating rotational speed profile, wherein the controller uses the rotational speed variation amplitude to control operation of the ventricular assist device so that a maximum rotational speed of the repeating rotational speed profile is greater than a minimum rotational speed of the repeating rotational speed profile by the rotational speed variation amplitude.

14. The circulatory support system of claim 13, wherein:
the rotational speed variation amplitude is set to be equal to a first rotational speed variation amplitude at a first reference heart rate;
the rotational speed variation amplitude is set to be equal to a second rotational speed variation amplitude at a second reference heart rate;
the second rotational speed variation amplitude is greater than the first rotational speed variation amplitude; and
the second reference heart rate is greater than the first reference heart rate.

15. The circulatory support system of claim 10, wherein each cycle of the repeating rotational speed profile generates a pressure pulse that is synchronized with ventricular systole of the respective cardiac cycle of the heart.

16. The circulatory support system of claim 10, wherein each cycle of the repeating rotational speed profile generates a pressure pulse that is synchronized with ventricular diastole of the respective cardiac cycle of the heart.

17. The circulatory support system of claim 9, wherein the non-transitory instructions are executable by the at least one processor to further cause the at least one processor to determine a rotational speed variation amplitude for the repeating rotational speed profile, wherein the controller uses the rotational speed variation amplitude to control operation of the ventricular assist device so that a maximum rotational speed of the repeating rotational speed profile is greater than a minimum rotational speed of the repeating rotational speed profile by the rotational speed variation amplitude.

18. The circulatory support system of claim 17, wherein:
the rotational speed variation amplitude is set to be equal to a first rotational speed variation amplitude at a first reference heart rate;
the rotational speed variation amplitude is set to be equal to a second rotational speed variation amplitude at a second reference heart rate;
the second rotational speed variation amplitude is greater than the first rotational speed variation amplitude; and
the second reference heart rate is greater than the first reference heart rate.

19. The circulatory support system of claim 1, wherein the non-transitory instructions are executable by the at least one processor to cause the at least one processor to:
process the electrocardiogram signal to detect an arrhythmia of the heart; and
output an arrhythmia alarm in response to detecting the arrhythmia of the heart.

20. The circulatory support system of claim 1, wherein the non-transitory instructions are executable by the at least one processor to cause the at least one processor to:
process the electrocardiogram signal to determine whether a heart rate of the patient is stable or unstable; and
reduce a rotation rate of the ventricular assist device in response a determination of an unstable heart rate.

21. The circulatory support system of claim 1, wherein the non-transitory instructions are executable by the at least one processor to cause the at least one processor to:
process the electrocardiogram signal to determine whether a heart rate of the patient has increased from a previous period;
process the electrocardiogram signal to determine whether a respiration rate has increased from the previous period; and
increase a rotational rate of the ventricular assist device in response to determining that each of the heart rate and the respiration rate has increased from the previous period.

22. The circulatory support system of claim 1, wherein:
the controller is configured to be implanted; and
the controller comprises the electrocardiogram electrodes.

23. The circulatory support system of claim 22, wherein each of the electrocardiogram electrodes form an external surface of the controller.

24. The circulatory support system of claim 1, further comprising an implantable cardiac monitor that comprises the electrocardiogram electrodes.

25. The circulatory support system of claim 1, further comprising an implantable transcutaneous energy transmission receiver that comprises the electrocardiogram electrodes.

26. The circulatory support system of claim 1, further comprising an implantable transcutaneous energy transmission receiver that comprises one of the electrocardiogram electrodes, and wherein the controller is configured to be implanted and comprises one of the electrocardiogram electrodes.
